(19) United States
(12) Patent Application Publication
(10) Pub. No.: US 2007/0264600 A1
(43) Pub. Date: Nov. 15, 2007

(54) Heterocyclic Colorants Based on Diazabenzoisoindoles

(76) Inventors: Heino Heckmann, Liederbach (DE); Hans Joachim Metz, Darmstadt (DE)

Correspondence Address: CLARIANT CORPORATION;INTELLECTUAL PROPERTY DEPARTMENT, 4000 MONROE ROAD, CHARLOTTE, NC, 28205, US

(21) Appl. No.: 10/560,095

(22) PCT Filed: May 21, 2004
(86) PCT No.: PCT/EP04/05459

§ 371 Date: Mar. 23, 2007

(30) Foreign Application Priority Data
Jun. 11, 2003 (DE) 103 26 211.3

(51) Int. Cl. G03C 5/30; C07D 239/70; C07D 403/04; C07D 471/04; C09D 11/00; G02B 6/036

(52) U.S. Cl. 430/399; 106/31.29; 106/31.47; 385/145; 544/250; 544/296; 544/345

(57) ABSTRACT

The invention is directed to novel colorants of formula (I), wherein A represents a group of general formulas (II), (III), (IV) or (V), and B represents a substituted or unsubstituted ortho-C6-C18 arylene, wherein C and D represent an alicyclic or heteroyclic group.

Within the field of color pigments there is a constant market demand for novel hues possessing high migration, light, and solvent fastnesses, good thermal stabilities, and high coloring intensity.

The subject matter claimed in U.S. Pat. No. 4,166,179 includes pigments deriving from 4,7-diazaisoindole, but there is no description of additional benzo-fusing on the indole framework.

The object was to find new colorants for dyeing or pigmenting organic or inorganic materials of high or low molecular weight, in particular high molecular weight organic materials, which are based on readily available intermediates.

It has been found that this object is achieved, surprisingly, by means of compounds of the formula (I).

In one aspect the present invention accordingly provides compounds of the general formula (I) 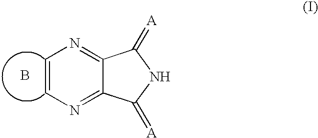
in which A is a group of the general formula (II), (III), (IV) or (V) 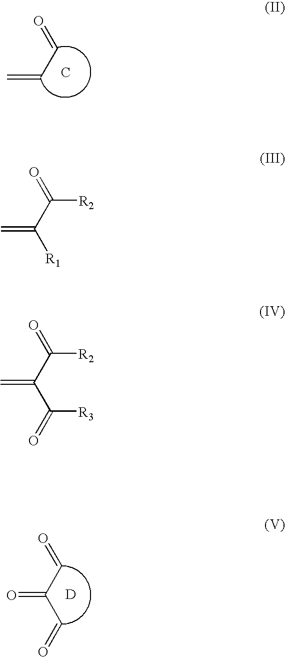
in which C and D are an alicyclic or heterocyclic group;

R₁ is CN or is a 5- to 7-membered heteroaromatic radical having 1, 2 or 3 heteroatoms from the group N, O, and S,

and R₂ and R₃ independently of one another are C₁-C₂₅ alkyl, C₅-C₁₂ cycloalkyl, C₆-C₂₄ aryl, OH, OR₄ or NR₄R₅, in which R₄ and R₅ are identical or different and are hydrogen, C₁-C₂₅ alkyl, C₅-C₁₂ cycloalkyl, C₆-C₂₄ aryl which is unsubstituted or substituted by 1, 2, 3 or 4 radicals halogen, R⁰, OR⁰, SR⁰, NH₂, NHR⁰, NR⁰₂, NO₂, COOH, COOR⁰, CONH₂, CONHR⁰, CONR⁰₂, CN, SO₃H, SO₂(OR⁰), SO₂R⁰, SO₂NHR⁰, SO₂NR⁰₂ or by a 5- to 7-membered heteroaromatic radical having 1, 2 or 3 heteroatoms from the group N, O, and S, or are a 5- to 7-membered heteroaromatic radical having 1, 2 or 3 heteroatoms from the group N, O, and S, R⁰ being C₁-C₁₈ alkyl or C₆-C₂₄ aryl;

and B is unsubstituted or mono- to tetrasubstituted ortho-C₆-C₁₈ arylene, suitable substituents being preferably halogens, R⁰, OR⁰, SR⁰, NH₂, NHR⁰, NR⁰₂, NO₂, COOH, COOR⁰, CONH₂, CONHR⁰, CONR⁰₂, CN, SO₃H, SO₂(OR⁰), SO₂R⁰, SO₂NHR⁰, SO₂NR⁰₂ or a 5- to 7-membered heteroaromatic radical having 1, 2 or 3 heteroatoms from the group N, O, and S.

Preference is further given to compounds of the formula (I), in which B is ortho-phenylene or 2,3-naphthylene.

Particularly preferred are compounds of the formula (I) in which A is a divalent alicyclic or heterocyclic radical of the formulae (a) to (g)

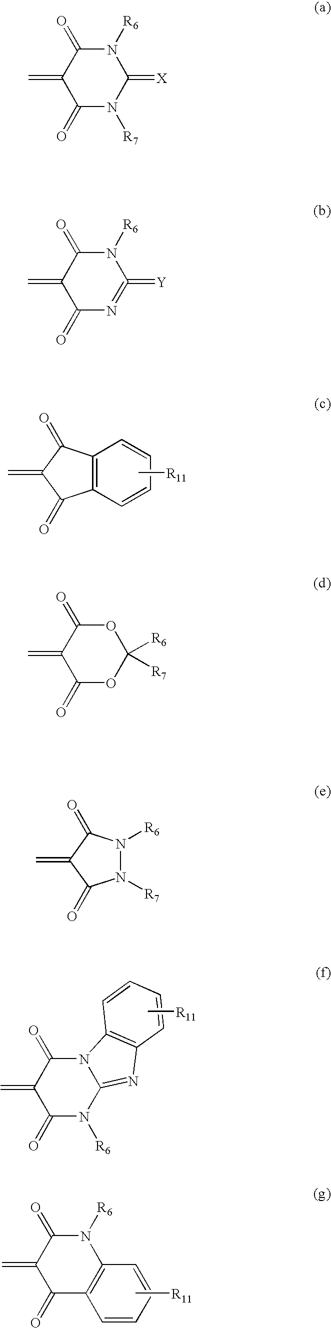

where R₆ and R₇ independently of one another are hydrogen, C₁-C₂₅ alkyl, C₅-C₁₂ cycloalkyl, C₆-C₂₄ aryl, C₁-C₂₅ alkyl(C₆-C₁₀ aryl), a 5- to 7-membered heteroaromatic radical having 1, 2 or 3 heteroatoms from the group N, O, and S, —(CH₂)ₙ—COR₈ or —(CH₂)ₘ—OR₉, in which R₈ is hydroxyl, amino, unsubstituted or mono- or polyhydroxyl- or -amino-substituted C₁-C₂₅ alkoxy, C₁-C₂₅ alkylamino, di(C₁-C₂₅ alkyl)amino,

C₁-C₂₅ alkyl(C₆-C₁₀ aryl)amino, (C₆-C₂₄ aryl)amino, di(C₆-C₂₄ aryl)amino, C₁-C₂₅ alkyl(C₆-C₁₀ aryl)amino, or C₂-C₂₄ alkenyloxy,

and R₉ is hydrogen or —CO—(C₁-C₂₅ alkyl), and n and m independently of one another are an integer from 0 to 6, preferably 1 to 4, and in which in R₆, R₇, R₆, and R₉ it is also possible for a C—C unit to be replaced by an ether unit C—O—C,

X is ═O, ═S or ═NR₁₀, in which R₁₀ has one of the definitions of R₆,

Y is hydrogen, R₇, OR₇, SR₇, NHCN or NR₇R₁₀,

and R₁₁ is hydrogen, halogen, CN, R₇, OR₇, SR₇, NR₇R₁₀, NO₂, SO₂(OR₇), SO₂R₇, SO₂NHR₇, SO₂N(R₇)₂ or PO₂(OR₇).

With particular preference R₆ and R₇ are hydrogen, C₁-C₁₈ alkyl, C₅-C₆ cycloalkyl, C₆-C₁₀ aryl, benzyl, pyridyl, pyrryl, thienyl, imidazolyl, oxazolyl, thiazolyl, pyrimidyl, hydroxycarbonyl-C₀-C₆ alkyl, C₁-C₁₈ alkoxycarbonyl-C₀-C₆ alkyl, aminocarbonyl-C₀-C₆ alkyl, C₁-C₁₈ alkylaminocarbonyl-C₀-C₆ alkyl, C₆-C₁₀ arylaminocarbonyl-C₀-C₆ alkyl, di(C₁-C₁₈ alkyl)aminocarbonyl-C₀-C₆ alkyl, C₁-C₁₈ alkyl-C₆-C₁₀ arylaminocarbonyl-C₀-C₆ alkyl and di(C₆-C₁₀ aryl)aminocarbonyl-C₀-C₆ alkyl.

With particular preference R₈ is hydroxyl, C₁-C₁₈ alkoxy, C₁-C₁₈ alkylamino, di(C₁-C₁₈ alkyl)amino, benzylamino, C₆-C₁₀ arylamino, di(C₆-C₁₀ aryl)amino or (C₂-C₁₈) alkenyloxy.

With particular preference R₁₁ is hydrogen, Cl, Br, C₁-C₁₈ alkyl, C₅-C₆ cycloalkyl, benzyl, C₆-C₁₀ aryl, pyridyl, pyrryl, thienyl, imidazolyl, oxazolyl, thiazolyl, pyrimidyl, C₁-C₁₈ alkoxy, C₆-C₁₀ aryloxy, C₁-C₁₈ alkylthio, C₆-C₁₀ arylthio, C₁-C₁₈ alkylamino, C₆-C₁₀ arylamino, di(C₁-C₁₈ alkyl)amino, C₁-C₁₈ alkyl(C₆-C₁₀ aryl)amino, di(C₆-C₁₀ aryl)amino, SO₃H, C₁-C₁₈ alkoxysulfonyl, C₁-C₁₈ alkylsulfonyl, C₁-C₁₈ alkylaminosulfonyl and di(C₁-C₁₈ alkyl)aminosulfonyl.

The present invention further provides a process for preparing compounds of the general formula (I) by reacting a 2,3-dicyanoquinoxaline of the formula (XIV) 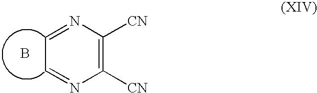
with a total of at least 2 equivalents of ammonia and/or alkoxides MOR₁₂, in which M is sodium or potassium, such as sodium methoxide, sodium ethoxide, sodium pentoxide, potassium methoxide or potassium tert-butoxide, to give di- or monoimino-substituted diazabenzoisoindoles bearing 0 to 2 alkoxy substituents, represented for example by compounds of the formulae (VI), (VII) or (VIII), 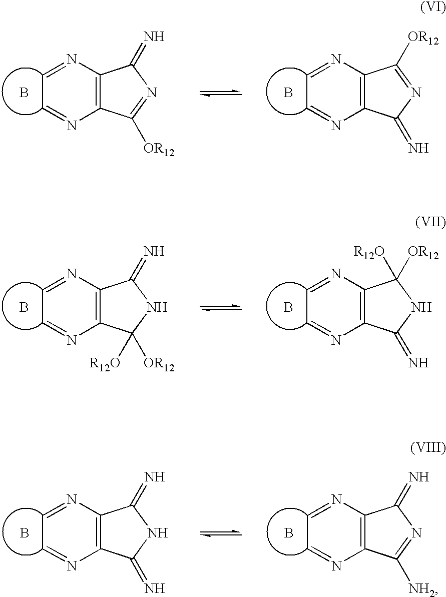
in which R₁₂ is C₁-C₁₈ alkyl or —(CH₂)ₘ—OH and m is an integer in the range from 1 to 6, and it is also possible for a C—C unit to be replaced by an ether unit C—O—C, in a solvent or solvent mixture under basic to neutral conditions at a temperature of −20 to 120° C., preferably 0 to 100° C., more preferably 20 to 80° C., which are subsequently reacted, following isolation or without isolation, in a solvent or solvent mixture under neutral to acidic conditions, preferably in the presence of an organic acid, such as formic acid, acetic acid or propionic acid, or of an inorganic acid, such as sulfuric acid, hydrochloric acid or phosphoric acid, and advantageously at a temperature of 10 to 250° C., in particular 20 to 200° C., more preferably 30 to 150° C., with at least 2 equivalents of a compound of the formulae (IX), (X), (XI) or (XII) 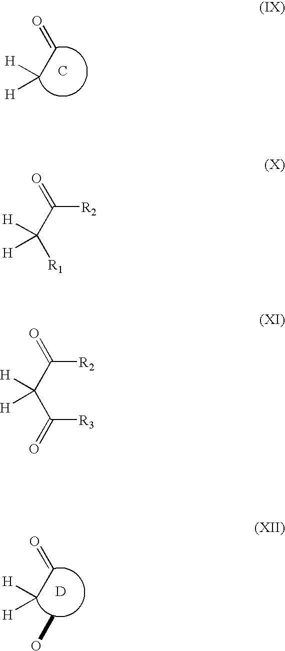
in which C, D, R₁, R₂, and R₃ are as defined above,

to give a further intermediate of the general formula (XIIIa) or (XIIIb) 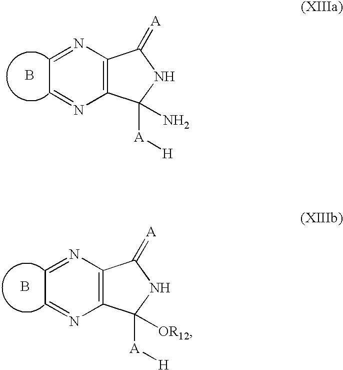
from which subsequently one mole of ammonia or HOR₁₂ is eliminated.

The elimination of ammonia and/or HOR₁₂ to give the compounds of the formula (I) according to the invention is accomplished advantageously at a temperature of −30 to 250° C., preferably −20 to 200° C., more preferably 0 to 150° C., preferably in the presence of a solvent such as water, methanol, ethanol, glycols, dimethylformamide, N-methylpyrrolidone or toluene, and more preferably in the presence of an organic acid, such as formic acid, acetic acid or propionric acid, or in the presence of an inorganic acid, such as sulfuric acid, hydrochloric acid or phosphoric acid.

2,3-Dicyanoquinoxalines of the formula (XIV) can be prepared in accordance with Liebigs Ann. Chem. 1981, 2, pages 333-341, by reacting corresponding 2,3-dichloroquinoxalines of the formula (XV) 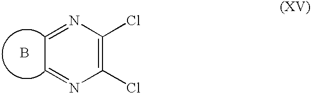
with at least twice the molar amount of tetraethylammonium cyanide in DMSO. Surprisingly it has now been found that a cyanide of a main-group or transition-group metal can also be used, in an organic solvent, in the presence of a phase-transfer catalyst, at elevated temperatures. Preferred cyanides are those of an alkali metal or alkaline earth metal, CuCN, Zn(CN)₂ or Fe(CN)₂, preferably sodium cyanide. The reaction of compounds of the general formula (XV) occurs preferably in an organic solvent having a high dielectric constant, such as DMSO, DMF, dimethylacetamide, NMP, acetonitrile, sulfolane, and dioxolane, preferably DMSO and NMP, in particular DMSO, in the presence of a phase-transfer catalyst selected from the group of quaternary alkylammonium salts, especially quaternary alkylammonium halides, such as distearyldimethylammonium chloride, trimethylbenzylammonium bromide or tetrabutylammonium chloride, at temperatures of 0 to 150° C., in particular at 10 to 100° C., preferably at 20 to 80° C.

The advantage of the new process is that it is possible to start from the relatively inexpensive sodium cyanide, which is less hygroscopic than the organic cyanide and, furthermore, has a significantly lower toxicity, and that the phase-transfer catalyst can be used in a substoichiometric molar amount, preferably in 0.01 to 0.9 times the molar amount, based on the starting product of the formula (XV).

The present invention additionally provides an intermediate of the general formula (XIIIa),

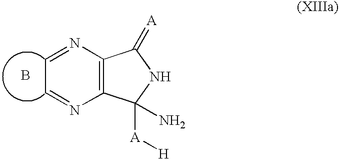

Compounds of the general formula (I) according to the present invention are used for dyeing or pigmenting organic or inorganic, high or low molecular weight, in particular high molecular weight organic, materials.

Depending on the identity of their substituents and of the high molecular weight organic material to be colored, the compounds of the invention can be used as polymer-soluble dyes or as pigments. In the latter case it is advantageous to convert the as-synthesized products (crude pigments) by after treatment in organic solvents—in which the pigments themselves are not dissolved—and at elevated temperatures, for example at 60 to 200° C., in particular at 70 to 150° C. and preferably at 75 to 100° C., into a finely disperse form often having further improved pigmentary properties. The aftertreatment is preferably combined with a grinding or kneading operation.

The colorants of the invention are outstandingly suitable for coloring high molecular weight materials, which may be organic or inorganic in nature, and are plastics and/or natural materials. These may be, for example, natural resins, drying oils, rubber or casein. Alternatively they may be modified natural materials, such as chlorinated rubber, oil-modified alkyd resins, viscose, cellulose derivatives, such as cellulose esters or cellulose ethers, and, in particular, synthetic organic polymers (plastics), which may be obtained by addition polymerization, polycondensation or polyaddition. From the class of the plastics prepared by addition polymerization mention may be made in particular of the following: polyolefins, such as polyethylene, polypropylene, polyisobutylene, and substituted polyolefins such as polystyrene, polyvinyl chloride, polyvinylidene chloride, polyvinylacetals, polyacrylonitrile, polyacrylic acid, polymethacrylic acid, polyacrylic esters and polymethacrylic esters or polybutadiene, for example, and also copolymers thereof.

From the class of the plastics prepared by polyaddition and polycondensation mention may be made of the following: polyesters, polyamides, polyimides, polycarbonates, polyurethanes, polyethers, polyacetals, and also the condensation products of formaldehyde with phenols (phenolic resins) and the condensation products of formaldehyde with urea, thiourea, and melamine (amino resins). The materials in question may also be silicones or silicone resins.

High molecular weight materials of this kind can be present individually or in mixtures in the form of plastic masses or melts or in the form of spinning solutions. They may also be present in the form of their monomers or in the polymerized state, in dissolved form, as film formers or binders for paints or printing inks, such as linseed-oil varnish, nitrocellulose, alkyd resins, melamine resins and formaldehyde resins or acrylic resins.

The compounds of the invention are suitable, accordingly, as colorants in oil-based or water-based paints, in coating materials of various kinds, camouflage paints, for spin coloring, for the mass coloring or pigmenting of plastics, in printing inks for the graphics industry, such as, for example, in paper, textile or decorative printing, and in the mass coloring of paper, for preparing inks, water-based or non-water-based ink-jet inks, microemulsion inks, and inks which operate in accordance with the hot-melt process.

The compounds of the invention are also suitable for use as colorants in electrophotographic toners and developers, such as, for example, one- or two-component powder toners (also called one- or two-component developers), magnetic toners, liquid toners, latex toners, polymerization toners, and specialty toners.

Typical toner binders are addition polymerization resins, polyaddition resins, and polycondensation resins, such as styrene, styrene-acrylate, styrene-butadiene, acrylate, polyester, phenol-epoxy resins, polysulfones, polyurethanes, individually or in combination, and also polyethylene and polypropylene, which may include further ingredients, such as charge control agents, waxes or flow assistants, or are modified subsequently with these additions.

The compounds of the invention are additionally suitable for use as colorants in powders and powder coating materials, particularly in triboelectrically or electrokinetically sprayable powder coating materials which are employed to coat the surfaces of articles made, for example, of metal, wood, plastic, glass, ceramic, concrete, textile material, paper or rubber.

Typical powder coating resins used include epoxy resins, carboxyl- and hydroxyl-containing polyester resins, polyurethane resins, and acrylic resins, together with customary hardeners. Resin combinations are also used. Thus, for example, epoxy resins are frequently used in combination with carboxyl- and hydroxyl-containing polyester resins. Typical hardener components (depending on the resin system) are, for example, acid anhydrides, imidazoles and also dicyandiamide and their derivatives, blocked isocyanates, bisacylurethanes, phenolic resins, melamine resins, triglycidyl isocyanurates, oxazolines, and dicarboxylic acids.

Additionally the compounds of the invention are suitable for use as colorants in inks, preferably ink-jet inks, for example water-based or nonwater-based ink-jet inks, microemulsion inks and also in those inks which operate in accordance with the hot-melt process.

Ink-jet inks contain in general a total of 0.5% to 15%, preferably 1.5% to 8%, by weight (calculated on a dry basis) of one or more of the compounds of the invention.

Microemulsion inks are based on organic solvents, water and, if desired, an additional hydrotropic substance (interface mediator).

Microemulsion inks contain 0.5% to 15%, preferably 1.5% to 8%, by weight of one or more of the compounds of the invention, 5% to 99% by weight of water and 0.5% to 94.5% by weight of organic solvent and/or hydrotropic compound.

Solvent-based ink-jet inks contain preferably 0.5% to 15% by weight of one or more of the compounds of the invention, 85% to 99.5% by weight of organic solvent and/or hydrotropic compounds.

Hot-melt inks are based mostly on waxes, fatty acids, fatty alcohols or sulfonamides which are solid at room temperature and liquefy on heating, the preferred melting range being situated between about 60° C. and about 140° C. Hot-melt ink-jet inks are composed, for example, essentially of 20% to 90% by weight of wax and 1% to 10% by weight of one or more of the compounds of the invention. Additionally there may be 0% to 20% by weight of an additional polymer (as “dye dissolver”), 0% to 5% by weight of dispersing assistants, 0% to 20% by weight of viscosity modifiers,; 0% to 20% by weight of plasticizers, 0% to 10% by weight of tack additive, 0% to 10% by weight of transparency stabilizer (which prevents, for example, the waxes crystallizing) and 0% to 2% by weight of antioxidant.

The colorants of the invention are also suitable, moreover, for use as colorants for color filters, both for additive and for subtractive color generation, and also as colorants for electronic inks (“e-inks”) or electronic paper (“e-paper”).

In the production of what are called color filters, both reflective and transparent color filters, pigments in the form of a paste or as pigmented photoresists in suitable binders (acrylates, acrylic esters, polyimides, polyvinyl alcohols, epoxides, polyesters, melamines, gelatin, caseins) are applied to the respective LCD components (e.g., TFT-LCD=thin film transistor liquid crystal displays or, e.g., ((S) TN-LCD=(super) twisted nematic-LCD). Besides high thermal stability, high pigment cleanness is a prerequisite for a stable paste or a pigmented photoresist.

Furthermore, the pigmented color filters may also be applied by ink-jet printing processes or other suitable printing processes.

The present invention provides, moreover, for the use of the colorants of the invention in optical layers for optical data storage, preferably for optical data storage where a laser is used to write the data. The solubility of the colorants in the application medium, which is needed for this application, can be adjusted by means of the identity and number of the substituents.

The compounds of the invention are, furthermore, suitable for use as colorants in cosmetics, for coloring seed, and for coloring mineral oils, lubricating greases, and waxes.

Depending on the nature of the substituents of the compounds of the invention the colorations obtained are distinguished by good heat fastness, light fastness and weather fastness, by chemical resistance and by the very good applications properties, e.g., crystallization fastness and dispersion fastness, and in particular, by their migration fastness, bleed fastness, fastness to overcoating, and solvent fastness. The compounds used as polymer-soluble dyes naturally have only little, or restricted, solvent fastness.

The invention additionally provides a composition comprising an organic or inorganic, high or low molecular weight material, particularly high molecular weight organic material, and at least one compound of the invention in a coloristically effective amount, generally in the range from 0.005% to 70% by weight, in particular from 0.01 % to 10% by weight, based on the organic or inorganic material.

### EXAMPLES

Intermediates

### Example A

### 2,3-Dicyanoquinoxaline

A mixture of 10.0 g of 2,3-dichloroquinoxaline, 5.40 g of sodium cyanide and 9.32 g of benzyltrimethylammonium chloride is stirred in 500 ml of DMSO at room temperature for 17 hours. With intensive stirring the reaction mixture is poured onto 1000 ml of ice-water, stirred for an hour, and filtered with-suction, and the solid product is washed with water. Drying at 60° C. gives 8.03 g (89% of theory) of pale gray crystals with the following formula

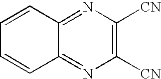

MS (m/e): 181 [M+H]⁺, 203 [M+Na]⁺

H NMR (DMSO): 8.34 (m, 2H), 8.23 (m, 2H)

### Example B

### 2,3-Dicyano-6-methoxyquinoxaline

A mixture of 31.1 g of 2,3-dichloro-6-methoxyquinoxaline, 14.7 g of sodium cyanide and 2.79 g of benzyltrimethylammonium chloride is stirred in 210 ml of DMSO at room temperature for 48 hours. With intensive stirring the reaction mixture is poured onto 520 ml of ice-water, stirred for an hour, and filtered with suction, and the solid product is washed with water. Drying at 40° C. gives 23.8 g (83% of theory) of pale gray crystals with the following formula

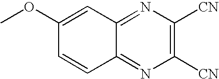

MS (m/e): 211 [M+H]⁺, 233 [M+Na]⁺

H NMR (DMSO): 8.21 (d, 1H), 7.83 (dd, 1H), 7.68 (d, 1H), 4.02 (s, 3H)

### Example C

### 6-Chloro-2,3-dicyanoquinoxaline

A mixture of 11.7 g of 2,3,6-trichloroquinoxaline, 5.39 g of sodium cyanide and 2.04 g of benzyltrimethylammonium chloride is stirred in 200 ml of DMSO at room temperature for 24 hours. With intensive stirring the reaction mixture is poured onto 520 ml of ice-water, stirred for an hour, and filtered with suction, and the solid product is washed with water. Drying at 40° C. gives 8.12 g (76% of theory) of a gray powder of a compound with the following formula

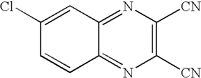

MS (m/e): 215 [M+H]⁺, 237 [M+Na]⁺

H NMR (DMSO): 8.53 (d, 1H), 8.37 (d, 1H), 8.26 (dd,1H)

### Example D

### 1-Amino-1-(2,4,6-trioxotetrahydropyrimidin-5-yl)-3-(2,4,6-trioxo-tetrahydropyrimidin-5-ylidene)-4,9-diazabenzo[f]isoindole

A suspension of 175 g of 2,3-dicyanoquinoxaline in 1700 ml of methanol is admixed dropwise at 10° C. with 23.0 g of sodium methoxide solution (30% in methanol) and stirred at room temperature for 16 hours. The mixture is diluted with 3500 ml of methanol and, following addition of 52.2 g of glacial acetic acid and 261 g of barbituric acid, is stirred at reflux for 6 hours. After cooling to room temperature the suspension is filtered and the solid product is washed with methanol and then with water and dried at 60° C. This gives 154 g (84% of theory) of a light brown powder of the following formula

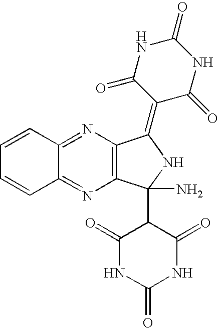

Melting point: >300° C.

MS (m/e): 437 [M+H]⁺, 459 [M+Na]⁺

### Example E

### 1-Amino-1-(1,3-dimethyl-2,4,6-trioxotetrahydropyrimidin-5-yl)-3-(1,3-dimethyl-2,4,6-trioxotetrahydropyrimidin-5-ylidene)-4,9-diazabenzo[f]isoindole

A suspension of 22.8 g of 2,3-dicyanoquinoxaline in 180 ml of methanol is admixed dropwise at 10° C. with 3.0 g of sodium methoxide solution (30% in methanol) and stirred at room temperature for 16 h. The mixture is diluted with 200 ml of methanol and, following addition of 6.8 g of glacial acetic acid and 41.6 g of 1,3-dimethylbarbituric acid, is stirred at reflux for 6 hours. After cooling to room temperature the suspension is filtered and the solid product is washed with methanol and then with water and dried at 60° C. This gives 33.3 g (54% of theory) of a beige-brown powder of the following formula

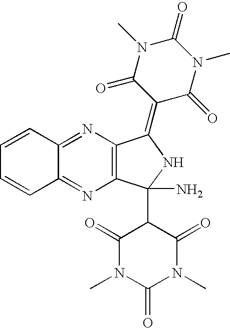

Melting point: >300° C.

MS (m/e): 493 [M+H]⁺, 515 [M+Na]⁺

### Example F

### 1-Amino-1-(1,3-dioxoindan-2-yl)-3-(1,3-dioxoindan-2-ylidene)-4,9-diazabenzo[f]isoindole

A suspension of 17.5 g of 2,3-dicyanoquinoxaline in 170 ml of methanol is admixed dropwise at 10° C. with 2.3 g of sodium methoxide solution (30% in methanol) and stirred at room temperature for 16 hours. The mixture is diluted with 360 ml of methanol and, following addition of 5.2 g of glacial acetic acid and 29.8 g of 1,3-dioxoindane, is stirred at reflux for 6 hours. After cooling to room temperature the suspension is filtered and the solid product is washed with methanol and then with water and dried at 60° C. This gives 31.5 g (69% of theory) of a brown powder of the following formula

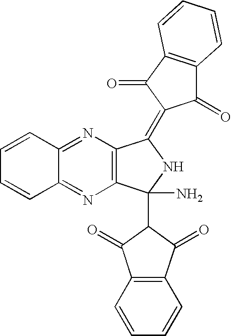

Melting point: >300° C.

MS (m/e): 473 [M+H]⁺, 495 [M+Na]⁺

### Example G

### 1-Amino-1-(2,4-dioxo-10H-benzo[4,5]imidazo[1,2-a]pyrimidin-3-yl)-3-(2,4-dioxo-10H-benzo[4,5]imidazo[1,2-a]pyrimidin-3-ylidene)-4,9-diazabenzo[f]isoindole

A suspension of 10.0 g of 2,3-dicyanoquinoxaline in 100 ml of methanol is admixed dropwise at 10° C. with 1.1 g of sodium methoxide solution (30% in methanol) and stirred at room temperature for 16 hours. The mixture is diluted with 200 ml of methanol and, following addition of 2.5 g of glacial acetic acid and 23.5 g of 1H-benzo[4,5]imidazo[1,2-a]pyrimidine-2,4-dione, is stirred at reflux for 6 hours. After cooling to room temperature the suspension is filtered and the solid product is washed with methanol and then with water and dried at 60° C. This gives 25.9 g (80% of theory) of a light brown powder of the following formula

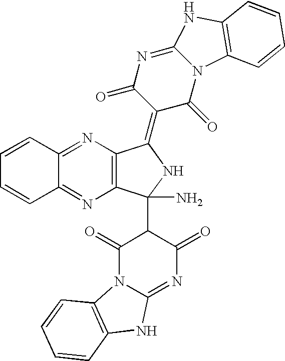

Melting point: >300° C.

MS (m/e): 583 [M+H]⁺, 605 [M+Na]⁺

Colorants

### Example 1

### 1,3-Bis(2,4,6-trioxotetrahydropyrimidin-5-ylidene)-4,9-diazabenzo[f]isoindole

137 g of 1-amino-1-(2,4,6-trioxotetrahydropyrimidin-5-yl)-3-(2,4,6-trioxo-tetrahydropyrimidin-5-ylidene)-4,9-diazabenzo[f]isoindole are stirred in 2060 ml of sulfuric acid (80%) at room temperature for 30 minutes. Over the course of 15 minutes and at a uniform rate this solution is added dropwise with stirring to 4900 ml of water (20° C.) and left to stand for a further 30 minutes. The suspension is filtered and the solid product is washed to neutrality with water and dried at 60° C. This gives 129 g (98% of theory) of a yellow compound of the following formula

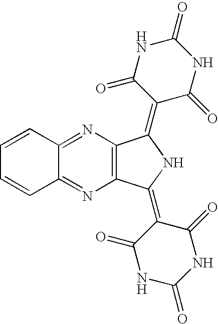

Melting point: >300° C.

MS (m/e): 420 [M+H]⁺, 442 [M+Na]⁺

### Example 2

### 1,3-Bis(1,3-dimethyl-2,4,6-trioxotetrahydropyrimidin-5-ylidene)-4,9-diazabenzo[f]isoindole

24 g of 1-amino-1-(1,3-dimethyl-2,4,6-trioxotetrahydropyrimidin-5-yl)-3-(1,3-dimethyl-2,4,6-trioxo-tetrahydropyrimidin-5-ylidene)-4,9-diazabenzo[f]isoindole are stirred in 480 ml of concentrated sulfuric acid at room temperature for 30 minutes. Over the course of 15 minutes and at a uniform rate this solution is added dropwise with stirring to 4800 ml of water and left to stand for a further 30 minutes. The suspension is filtered and the solid product is washed to neutrality with water and dried at 60° C. This gives 16.7 g (72% of theory) of a yellow compound of the following formula

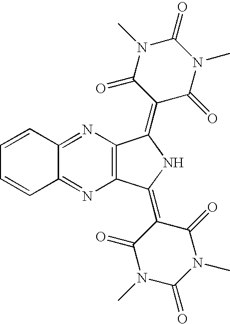

Melting point: >300° C.

MS (m/e): 476 [M+H]⁺, 498 [M+Na]⁺

### Example 3

### 1,3-Bis(1,3-dioxoindan-2-ylidene)-4,9-diazabenzo[f]isoindole

5.0 g of 1-amino-1-(1,3-dioxoindan-2-yl)-3-(1,3-dioxoindan-2-ylidene)-4,9-diazabenzo-[f]isoindole are stirred in 75 ml of concentrated sulfuric acid at room temperature for 30 minutes. Over the-course of 15 minutes and at a uniform rate this solution is added dropwise with stirring to 180 ml of water and left to stand for a further 30 minutes. The suspension is filtered and the solid product is washed to neutrality with water and dried at 60° C. This gives 4.49 g (93% of theory) of a reddish yellow compound of the following formula

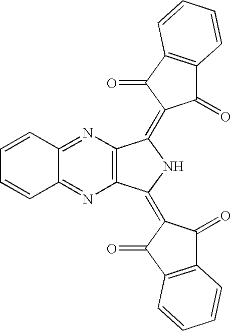

Melting point: >300° C.

MS (m/e): 476 [M+H]⁺, 498 [M+Na]⁺

### Example 4

### 1,3-Bis(2,4-dioxo-10H-benzo[4,5]imidazo[1,2-a]pyrimidin-3-ylidene)-4,9-diazabenzo[f]isoindole

5.0 g of 1-amino-1-(2,4-dioxo-10H-benzo[4,5]imidazo[1,2-a]pyrimidin-3-yl)-3-(2,4-dioxo-10H-benzo[4,5]imidazo[1,2-a]pyrimidin-3-ylidene)-4,9-diazabenzo[f]isoindole are stirred in 75 ml of concentrated sulfuric acid at room temperature for 30 minutes. Over the course of 15 minutes and at a uniform rate this solution is added dropwise with stirring to 180 ml of water and left to stand for a further 30 minutes. The suspension is filtered and the solid product is washed to neutrality with water and dried at 60° C. This gives 4.14 g (85% of theory) of a yellowish brown compound of the following formula

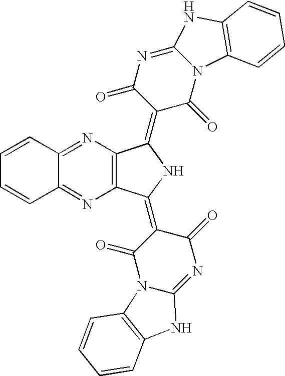

Melting point: >300° C.

MS (m/e): 566 [M+H]⁺, 588 [M+Na]⁺

### Application Examples

To assess the properties of the inventively prepared pigments in the coatings sector a selection was made, from among the multiplicity of known varnishes, of an aromatics-containing alkyd-melamine resin varnish (AM) based on a medium-oil alkyd resin and on a butanol-etherified melamine resin, and also of an aromatics-free, air-drying alkyd resin varnish (LA) based on a long-oil soya alkyd resin.

### Application Example 1

Application of the pigment from Example 1 in AM varnish produces strong greenish yellow coatings in the masstone and in the white reduction.

### Application Example 2

Application of the pigment from Example 2 in LA varnish produces strong reddish yellow coatings in the masstone and in the white reduction.

### Application Example 3

Application of the pigment from Example 3 in LA varnish produces strong reddish yellow coatings in the masstone and in the white reduction.

### Application Example 4

Application of the pigment from Example 4 in LA varnish produces reddish yellow coatings in the masstone and in the white reduction.

1. A compound of the formula (I) 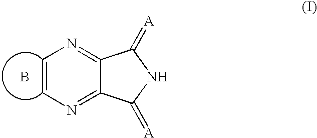
wherein A is a group of the general formula (II), (III), (IV) or (V) 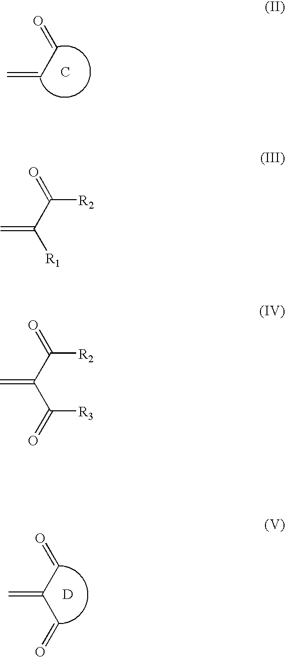
wherein C and D are an alicyclic or heterocyclic group;
R₁ is CN or is a 5- to 7-membered heteroaromatic radical having 1, 2 or 3 heteroatoms selected from the group consisting of N, O, and S,
and R₂ and R₃ independently of one another are C₁-C₂₅ alkyl, C₅-C₁₂ cycloalkyl, C₆-C₂₄ aryl, OH, OR₄ or NR₄R₅, wherein R₄ and R₅ are identical or different and are hydrogen, C₁-C₂₅ alkyl, C₅-C₁₂ cycloalkyl, C₆-C₂₄ aryl unsubstituted or substituted by 1, 2, 3 or 4 radicals halogen, R⁰, OR⁰, SR⁰, NH₂, NHR⁰, NR⁰₂, NO₂, COOH, COOR⁰, CONH₂, CONHR⁰, CONR⁰₂, CN, SO₃H, SO₂(OR⁰), SO₂R⁰, SO₂NHR⁰, SO₂NR⁰₂ a 5- to 7-membered heteroaromatic radical having 1, 2 or 3 heteroatoms selected from the group consisting of N, O, and S, or are a 5- to 7-membered heteroaromatic radical having 1, 2 or 3 heteroatoms selected from the group consisting of N, O, and S,
R⁰ is C₁-C₁₈ alkyl or C₆-C₂₄ aryl;
and B is unsubstituted or mono- to tetrasubstituted ortho-C₆-C₁₈ arylene.

2. A compound as claimed in claim 1, wherein A is a divalent alicyclic or heterocyclic radical of the formulae (a) to (g) 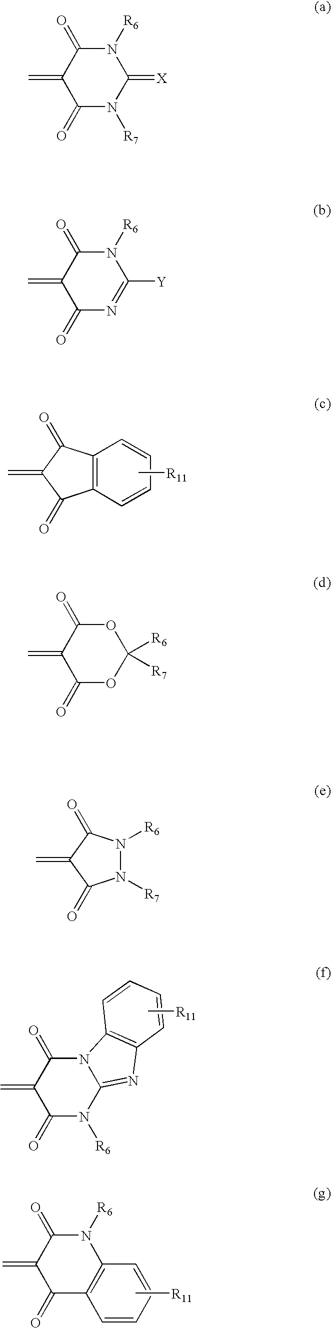
where R₆ and R₇ independently of one another are hydrogen, C₁-C₂₅ alkyl, C₅-C₁₂ cycloalkyl, C₆-C₂₄ aryl, C₁-C₂₅ alkyl(C₆-C₁₀ aryl), a 5- to 7-membered heteroaromatic radical having 1, 2 or 3 heteroatoms selected from the group consisting of N, O, S, —(CH₂)ₙ—COR₈ and —CH₂)ₘ—OR₉, wherein R₈ is hydroxyl, amino, unsubstituted or mono- or polyhydroxyl- or -amino-substituted C₁-C₂₅ alkoxy, C₁-C₂₅ alkylamino, di(C₁-C₂₅ alkyl)amino, C₁-C₂₅ alkyl(C₆-C₁₀ aryl)amino, (C₆-C₂₄ aryl)amino, di(C₆-C₂₄ aryl)amino, C₁-C₂₅ alkyl(C₆-C₁₀ aryl)amino, or C₂-C₂₄ alkenyloxy, and R₉ is hydrogen or —CO—(C₁-C₂₅ alkyl), and n and m independently of one another are an integer from 0 to 6, and wherein in R₆, R₇, R₈, and R₉, optionally a C—C unit is replaced by an ether unit C—O—C,
X is ═O, ═S or ═NR₁₀, wherein R₁₀ has one of the definitions of R₆,
Y is hydrogen, R₇, OR₇, SR₇, NHCN or NR₇R₁₀,
and R₁₁ is hydrogen, halogen, CN, R₇, OR₇, SR₇, NR₇R₁₀, NO₂, SO₂(OR₇), SO₂R₇, SO₂NHR₇, SO₂N(R₇)₂ or PO₂(OR₇).

3. A compound as claimed in claim 1, wherein R₆ and R₇ are hydrogen, C₁-C₁₈ alkyl, C₅-C₆ cycloalkyl, C₆-C₁₀ aryl, benzyl, pyridyl, pyrryl, thienyl, imidazolyl, oxazolyl, thiazolyl, pyrimidyl, hydroxycarbonyl-C₀-C₆ alkyl, C₁-C₁₈ alkoxycarbonyl-C₀-C₆ alkyl, aminocarbonyl-C₀-C₆ alkyl, C₁-C₁₈ alkylaminocarbonyl-C₀-C₆ alkyl, C₆-C₁₀ arylaminocarbonyl-C₀-C₆ alkyl, di(C₁-C₁₈ alkyl)aminocarbonyl-C₀-C₆ alkyl, C₁-C₁₈ alkyl-C₆-C₁₀ arylaminocarbonyl-C₀-C₆ alkyl or di(C₆-C₁₀ aryl)aminocarbonyl-C₀-C₆ alkyl.

4. A compound as claimed in claim 2, wherein R₈ is hydroxyl, C₁-C₁₈ alkoxy, C₁-C₁₈ alkylamino, di(C₁-C₁₈ alkyl)amino, benzylamino, C₆-C₁₀ arylamino, di(C₆-C₁₀ aryl)amino or (C₂-C₁₈) alkenyloxy.

5. A compound as claimed in claim 2, wherein R₁₁ is hydrogen, Cl, Br, C₁-C₁₈ alkyl, C₅-C₆ cycloalkyl, benzyl, C₆-C₁₀ aryl, pyridyl, pyrryl, thienyl, imidazolyl, oxazolyl, thiazolyl, pyrimidyl, C₁-C₁₈ alkoxy, C₆-C₁₀ aryloxy, C₁-C₁₈ alkylthio, C₆-C₁₀ arylthio, C₁-C₁₈ alkylamino, C₆-C₁₀ arylamino, di(C₁-C₁₈ alkyl)amino, C₁-C₁₈ alkyl(C₆-C₁₀ aryl)amino, di(C₆-C₁₀ aryl)amino, SO₃H, C₁-C₁₈ alkoxysulfonyl, C₁-C₁₈ alkylsulfonyl, C₁-C₁₈ alkylaminosulfonyl or di(C₁-C₁₈ alkyl)aminosulfonyl.

6. A process for preparing a compound as claimed in claim 1, comprising the steps of reacting, in a first reaction step a 2,3-dicyanoquinoxaline of the formula (XIV) 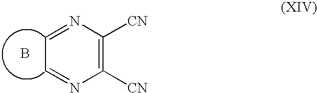
with a total of at least 2 equivalents of at least one of ammonia or alkoxides MOR₁₂, wherein M is sodium or potassium, to give di- or monoimino-substituted diazabenzoisoindoles of the formulae (VI), (VII) or (VIII) 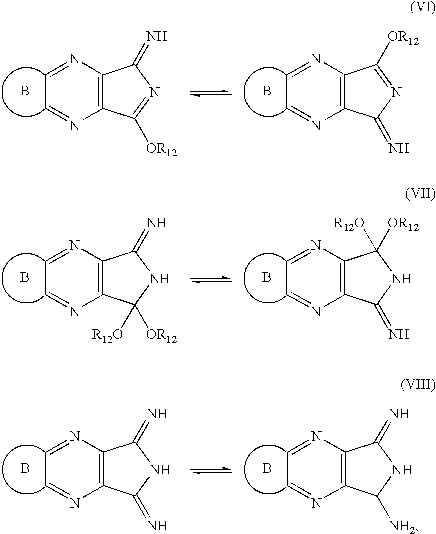
wherein R₁₂ is C₁-C₁₈ alkyl or —CH₂)ₘ—OH and m is an integer 1 to 6, and optionally a C—C unit is replaced by an ether unit C—O—C,
in a solvent or solvent mixture under basic to neutral conditions at a temperature of −20 to 120° C.,
reacting, in a second reacting step, the di- or monoimino-substituted diazabenzoisoindoles of the formulae (VI), (VII) or (VIII), in a solvent or solvent mixture under neutral to acidic conditions, with at least 2 equivalents of a compound of the formulae (IX), (X), (XI) or (XII) 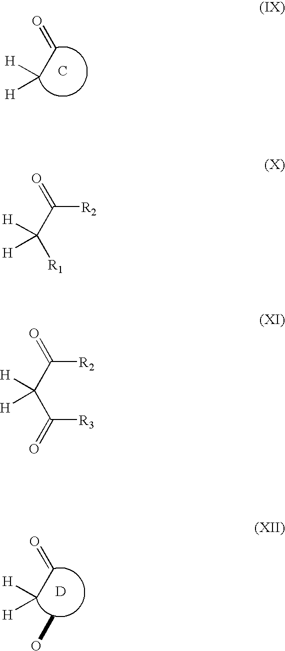
to give a further intermediate of the general formula (XIIIa) or (XIIIb) 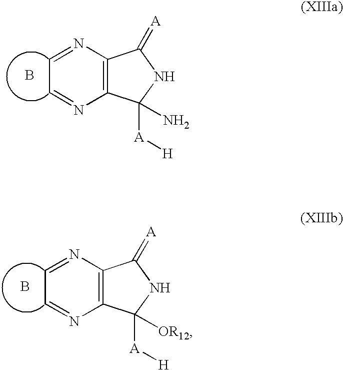
and eliminating one mole of ammonia or HOR₁₂ from the further intermediate.

7. The process as claimed in claim 6, wherein the 2,3-dicyanoquinoxaline is prepared by reacting 2,3-dichloroquinoxalines of the formula (XV) 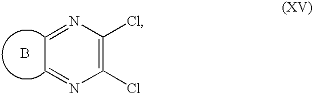
with a cyanide of a main-group or transition-group metal in an organic solvent in the presence of a phase-transfer catalyst at elevated temperatures.

8. A compound of the general formula (XIIIa), 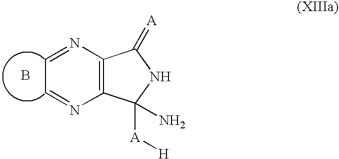
wherein A is a group of the general formula (II), (III), (IV) or (V) 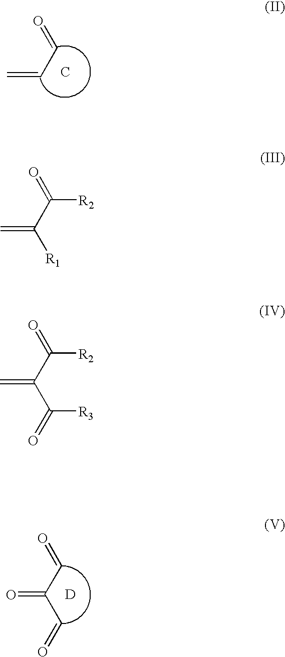
iwherein C and D are an alicyclic or heterocyclic group;
R₁ is CN or is a 5- to 7-membered heteroaromatic radical having 1, 2 or 3 heteroatoms selected from the group consisting of N, O, and S,
and R₂ and R₃ independently of one another are C₁-C₂₅ alkyl, C₅-C₁₂ cycloalkyl, C₆-C₂₄ aryl, OH, OR₄ or NR₄R₅, in which wherein R₄ and R₅ are identical or different and are hydrogen, C₁-C₂₅ alkyl C₅-C₁₂ cycloalkyl, C₆-C₂₄ aryl which is unsubstituted or substituted by 1, 2, 3 or 4 radicals halogen, R⁰, OR⁰, SR⁰, NH₂, NHR⁰, NR⁰₂, NO₂, COOH, COOR⁰, CONH₂, CONHR⁰, CONR⁰₂, CN, SO₃H, SO₂(OR⁰), SO₂R⁰, SO₂NHR⁰, SO₂NR⁰₂ or by a 5- to 7-membered heteroaromatic radical having 1, 2 or 3 heteroatoms selected from the group consisting of N, O, and S, or are a 5- to 7-membered heteroaromatic radical having 1, 2 or 3 heteroatoms selected from the group consisting of N, O, and S,
R⁰ being is C₁-C₁₈ alkyl or C₆-C₂₄ aryl;
and B is unsubstituted or mono- to tetrasubstituted ortho-C₆-C₁₈ arylene.

9. A process for coloring an organic or inorganic material of high or low molecular weight comprising the step of dyeing or pigmenting the organic or inorganic material with a compound as claimed in claim 1.

10. The process as claimed in claim 9, wherein the organic or inorganic material is selected from the group consisting of oil-based paints, water-based paints, coating materials, camouflage paints, plastics, printing inks, paper, seed, inks, water-based or non-water-based ink-jet inks, microemulsion inks, and inks which operate in accordance with the hot-melt process.

11. A colored composition comprising a compound as claimed in claim 1, wherein the composition is selected from the group consisting of electrophotographic toners, electrophotographic developers, color filters, electronic inks, powder coating materials, and optical layers for optical data storage.

12. A composition comprising an organic or inorganic, high or low molecular weight material and a compound as claimed in claim 1, wherein the compound is present in an amount of 0.005% to 70% by weight, based on the organic or inorganic material.
